(19) United States
(12) Patent Application Publication
(10) Pub. No.: US 2026/0215663 A1
(43) Pub. Date: Jul. 30, 2026

(54) ENDOSCOPE BENDING MEMBERS

(71) Applicant: SG ENDOSCOPY PTE LTD, Singapore (SG)

(72) Inventors: Jin Mei FAN, Singapore (SG); Kiyoshi CHIKASHIGE

(73) Assignee: SG ENDOSCOPY PTE LTD, Singapore (SG)

(21) Appl. No.: 19/147,680

(22) PCT Filed: Jan. 4, 2024
(86) PCT No.: PCT/SG2024/050010

§ 371 Date: Jul. 11, 2025

(30) Foreign Application Priority Data
Jan. 12, 2023 (SG) 10202300095S

(51) Int. Cl. A61B 1/005; A61B 1/00; A61B 1/008

(52) U.S. Cl. CPC A61B 1/0055; A61B 1/0011; A61B 1/008

(57) ABSTRACT

The present disclosure generally relates to an endoscope bending member (100). The bending member (100) comprises a longitudinal axis (110) and bending segments (120) successively connected along the longitudinal axis (110). Each bending segment (120) comprises a pair of proximal arcuate extensions (130), a proximal stopper element (132) circumferentially offset from the proximal arcuate extensions (130), a distal pivot extension (140), and a distal stopper element (142) circumferentially offset from the distal pivot extension (140). Adjacent bending segments (120) are engaged by the proximal arcuate extensions (130) and distal pivot extension (140) such that the bending segments (120) are rotatable to bend the bending member (100) about bending axes (112) perpendicular to the longitudinal axis (110). The proximal stopper elements (132) and distal stopper elements (142) of adjacent bending segments (120) are also engaged with each other to resist rotation of the bending segments (120) about the longitudinal axis (110).

### CROSS REFERENCE TO RELATED APPLICATIONS

The present disclosure claims the benefit of Singapore Patent Application 10202300095S filed on 12 Jan. 2023, which is incorporated in its entirety by reference herein.

### TECHNICAL FIELD

The present disclosure generally relates to endoscope bending members. More particularly, the present disclosure describes various embodiments of bending members for endoscopes, endoscopes comprising such bending members, as well as methods for manufacturing such bending members.

### BACKGROUND

Endoscopes are medical instruments used in various medical procedures such as gastrointestinal endoscopic examinations. For example, endoscopes are used in upper gastrointestinal endoscopy to diagnose and treat problems in the upper gastrointestinal tract. For example, endoscopes are used in ureteroscopy to diagnose and treat problems in the ureters and kidneys.

FIG. 1A shows an exemplary endoscope 10 comprising an operation handle 12 and an insertion tube 14 to be inserted into a patient's body, such as into the duodenum or ureters. To facilitate insertion of the insertion tube 14 into the patient and/or to reduce pain or injury to the patient, the insertion tube 14 includes a bending member 20 at a distal end 16 of the insertion tube 14. The bending member 20 is configured to perform bending actions by operation of one or more angulation wires using the operation handle 12, thereby positioning the insertion tube 14 at specific areas of the patient's body for observation.

Some conventional bending members 20A,20B for endoscopes are shown in FIGS. 1B and 1C. Each conventional bending member 20A,20B includes a plurality of bending segments 22A,22B, respectively, rotatably coupled to each other such that the respective conventional bending member 20A,20B can be bent at a predetermined angle in a predetermined direction. For example, the respective bending segments 22A,22B are coupled together using pivot pins.

When the conventional bending members 20A,20B are bent, they are subjected to forces such as bending, torsion, and tension. For example, when the bending members 20A,20B are inserted into narrow spaces such as the duodenum during upper gastrointestinal endoscopy or the ureters during ureteroscopy, there can be instances when the bending members 20A,20B become twisted due to torsion forces or even break.

FIGS. 1D to 1G show examples of the conventional bending members 20A,20B becoming damaged from torsion forces. Specifically, under high enough torsion forces, a respective bending segment 22Aa,22Ba would overcome and twist over a respective adjacent bending segment 22Ab,22Bb and damage the respective bending member 20A,20B. The damaged bending members 20A,20B would harm the affected part of the patient's body and it would be difficult or impossible for the bending members 20A,20B to return to their original neutral positions.

Endoscope bending members should thus have high resistance to these forces, specifically bending, torsion, and tension forces, in order to minimize the risk of damage or breakage of the bending members which would be dangerous or even fatal to the patient. Therefore, in order to address or alleviate at least one of the aforementioned problems and/or disadvantages, there is a need to provide improved endoscope bending members.

### SUMMARY

According to a first aspect of the present disclosure, there is a bending member for an endoscope. The bending member comprises:
a lumen extending therethrough;
a plurality of bending segments successively and rotatably connected to each other along a longitudinal axis through the lumen such that the bending member is bendable; and
each bending segment comprising:
   a pair of proximal arcuate extensions extending proximally from the bending segment;
   a proximal stopper element circumferentially offset about the longitudinal axis from the pair of proximal arcuate extensions; and
   a distal pivot extension extending distally from the bending segment; and
   a distal stopper element circumferentially offset about the longitudinal axis from the distal pivot extension,
wherein a first bending segment is engaged with a proximally adjacent bending segment and a distally adjacent bending segment, such that:
   the pair of proximal arcuate extensions of the first bending segment are engaged around the distal pivot extension of the proximally adjacent bending segment, such that the first and proximally adjacent bending segments are rotatable about a proximal bending axis through the distal pivot extension of the proximally adjacent bending segment;
   the pair of proximal arcuate extensions of the distally adjacent bending segment are engaged around the distal pivot extension of the first bending segment, such that the first and distally adjacent bending segments are rotatable about a distal bending axis through the distal pivot extension of the first bending segment;
   the proximal element of the first bending segment is engaged with the distal stopper element of the proximally adjacent bending segment to resist rotation of the first and proximally adjacent bending segments about the longitudinal axis; and
   the proximal stopper element of the distally adjacent bending segment is engaged with the distal stopper element of the first bending segment to resist rotation of the first and distally adjacent bending segments about the longitudinal axis; and
wherein bending segments are rotatable with respect to each other about the respective bending axes to thereby bend the bending member, the bending axes perpendicular to the longitudinal axis.

According to a second aspect of the present disclosure, there is a method for manufacturing a bending member for an endoscope. The method comprises:
laser cutting an elongated tube comprising a lumen extending therethrough;
forming a plurality of bending segments along the elongated tube from said laser cutting of the elongated tube, the bending segments successively and rotatably connected to each other along a longitudinal axis through the lumen such that the bending member is bendable; and
forming each bending segment of the plurality of bending segments from said laser cutting of the elongated tube, each bending segment comprising:
   a pair of proximal arcuate extensions extending proximally from the bending segment;
   a proximal stopper element circumferentially offset about the longitudinal axis from the pair of proximal arcuate extensions; and
   a distal pivot extension extending distally from the bending segment; and
   a distal stopper element circumferentially offset about the longitudinal axis from the distal pivot extension,
wherein a first bending segment is engaged with a proximally adjacent bending segment and a distally adjacent bending segment, such that:
   the pair of proximal arcuate extensions of the first bending segment are engaged around the distal pivot extension of the proximally adjacent bending segment, such that the first and proximally adjacent bending segments are rotatable about a proximal bending axis through the distal pivot extension of the proximally adjacent bending segment;
   the pair of proximal arcuate extensions of the distally adjacent bending segment are engaged around the distal pivot extension of the first bending segment, such that the first and distally adjacent bending segments are rotatable about a distal bending axis through the distal pivot extension of the first bending segment;
   the proximal element of the first bending segment is engaged with the distal stopper element of the proximally adjacent bending segment to resist rotation of the first and proximally adjacent bending segments about the longitudinal axis; and
   the proximal stopper element of the distally adjacent bending segment is engaged with the distal stopper element of the first bending segment to resist rotation of the first and distally adjacent bending segments about the longitudinal axis; and
wherein bending segments are rotatable with respect to each other about the respective bending axes to thereby bend the bending member, the bending axes perpendicular to the longitudinal axis.

According to a third aspect of the present disclosure, there is a bending member for an endoscope. The bending member comprises:
a lumen extending therethrough;
a plurality of bending segments successively and rotatably connected to each other along a longitudinal axis through the lumen such that the bending member is bendable; and
each bending segment comprising:
   a proximal pivot extension extending proximally from the bending segment;
   a pair of proximal arcuate extensions extending proximally from the bending segment, the proximal pivot extension disposed between
the pair of proximal arcuate extensions;
   an inner pair of distal arcuate extensions extending distally from the bending segment; and
   an outer pair of distal arcuate extensions extending distally from the bending segment, the inner pair of distal arcuate extensions disposed between the outer pair of distal arcuate extensions,
wherein a first bending segment is engaged with a proximally adjacent bending segment and a distally adjacent bending segment, such that:
   the inner pair of distal arcuate extensions of the first bending segment are engaged around the proximal pivot extension of the distally adjacent bending segment, the pair of proximal arcuate extensions of the distally adjacent bending segment are engaged around the inner pair of distal arcuate extensions of the first bending segment, and the outer pair of distal arcuate extensions of the first bending segment are engaged around the pair of proximal arcuate extensions of the distally adjacent bending segment, such that the first and distally adjacent bending segments are rotatable about a distal bending axis through the proximal pivot extension of the distally adjacent bending segment; and
   the inner pair of distal arcuate extensions of the proximally adjacent bending segment are engaged around the proximal pivot extension of the first bending segment, the pair of proximal arcuate extensions of the first bending segment are engaged around the inner pair of distal arcuate extensions of the proximally adjacent bending segment, and the outer pair of distal arcuate extensions of the proximally adjacent bending segment are engaged around the pair of proximal arcuate extensions of the first bending segment, such that the first and proximally adjacent bending segments are rotatable about a proximal bending axis through the proximal pivot extension of the first bending segment; and
wherein bending segments are rotatable with respect to each other about the respective bending axes to thereby bend the bending member, the bending axes perpendicular to the longitudinal axis.

According to a fourth aspect of the present disclosure, there is a method for manufacturing a bending member for an endoscope. The method comprises:
laser cutting an elongated tube comprising a lumen extending therethrough;
forming a plurality of bending segments along the elongated tube from said laser cutting of the elongated tube, the bending segments successively and rotatably connected to each other along a longitudinal axis through the lumen such that the bending member is bendable; and
   forming each bending segment of the plurality of bending segments from said laser cutting of the elongated tube, each bending segment comprising:
      a proximal pivot extension extending proximally from the bending segment;
      a pair of proximal arcuate extensions extending proximally from the bending segment, the proximal pivot extension disposed between the pair of proximal arcuate extensions;
      an inner pair of distal arcuate extensions extending distally from the bending segment; and
      an outer pair of distal arcuate extensions extending distally from the bending segment, the inner pair of distal arcuate extensions disposed between the outer pair of distal arcuate extensions,
   wherein a first bending segment is engaged with a proximally adjacent bending segment and a distally adjacent bending segment, such that:
      the inner pair of distal arcuate extensions of the first bending segment are engaged around the proximal pivot extension of the distally adjacent bending segment, the pair of proximal arcuate extensions of the distally adjacent bending segment are engaged around the inner pair of distal arcuate extensions of the first bending segment, and the outer pair of distal arcuate extensions of the first bending segment are engaged around the pair of proximal arcuate extensions of the distally adjacent bending segment, such that the first and distally adjacent bending segments are rotatable about a distal bending axis through the proximal pivot extension of the distally adjacent bending segment; and
      the inner pair of distal arcuate extensions of the proximally adjacent bending segment are engaged around the proximal pivot extension of the first bending segment, the pair of proximal arcuate extensions of the first bending segment are engaged around the inner pair of distal arcuate extensions of the proximally adjacent bending segment, and the outer pair of distal arcuate extensions of the proximally adjacent bending segment are engaged around the pair of proximal arcuate extensions of the first bending segment, such that the first and proximally adjacent bending segments are rotatable about a proximal bending axis through the proximal pivot extension of the first bending segment; and
   wherein bending segments are rotatable with respect to each other about the respective bending axes to thereby bend the bending member, the bending axes perpendicular to the longitudinal axis.

Endoscope bending members according to the present disclosure are thus disclosed herein. Various features and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A to 1G are illustrations of an endoscope comprising conventional bending members.

FIGS. 2A and 2B are illustrations of a first bending member for an endoscope, according to embodiments of the present disclosure.

FIGS. 3A and 3B are illustrations of bending segments of the first bending member.

FIGS. 4A and 4B are further illustrations of the bending segments of the first bending member.

FIGS. 5A and 5B are further illustrations of the first bending member.

FIGS. 6A and 6B are illustrations of a second bending member for an endoscope, according to embodiments of the present disclosure.

FIGS. 7A and 7B are illustrations of bending segments of the second bending member.

FIGS. 8A and 8B are illustrations of engaged bending segments of the first and second bending members.

FIG. 9 is a flowchart illustration of a method for manufacturing a bending member for an endoscope, according to embodiments of the present disclosure.

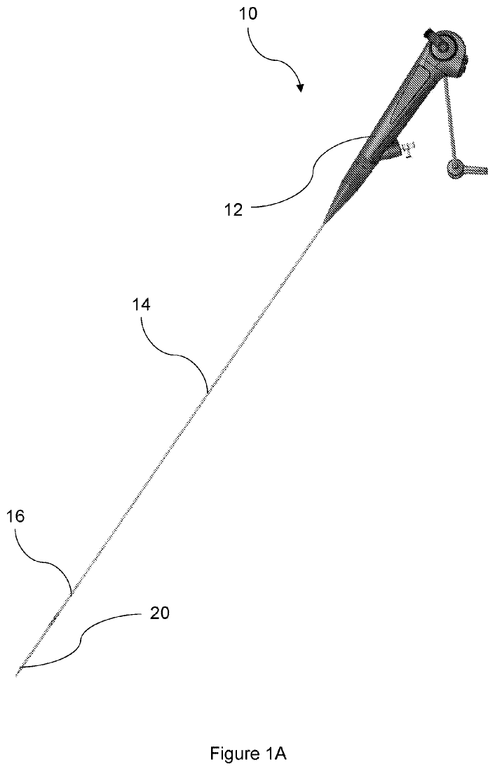

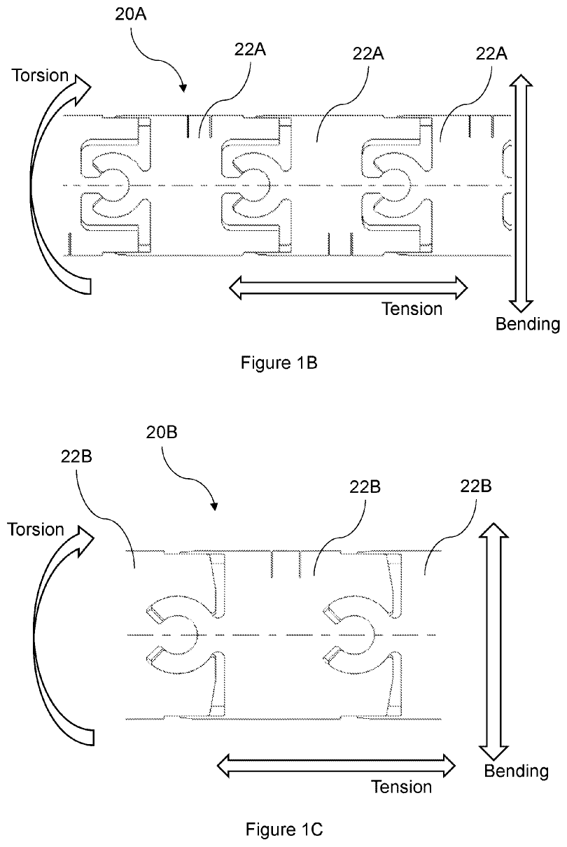

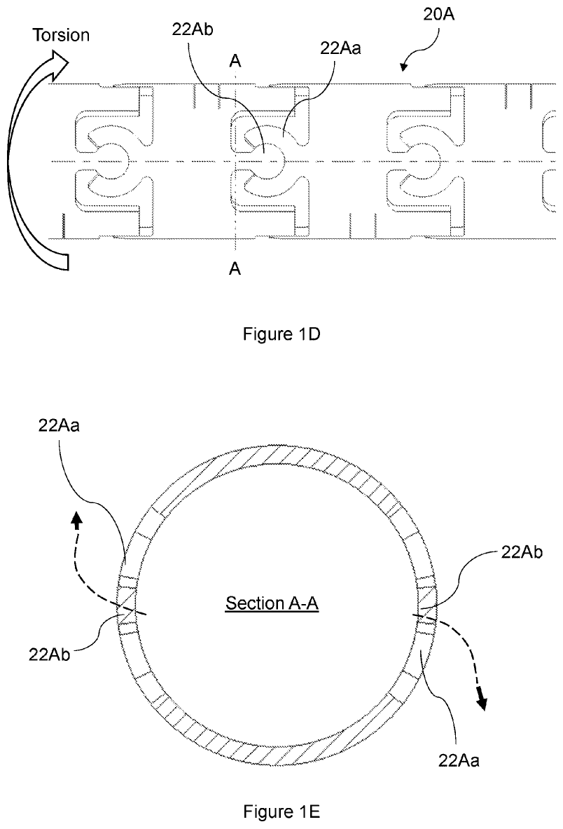

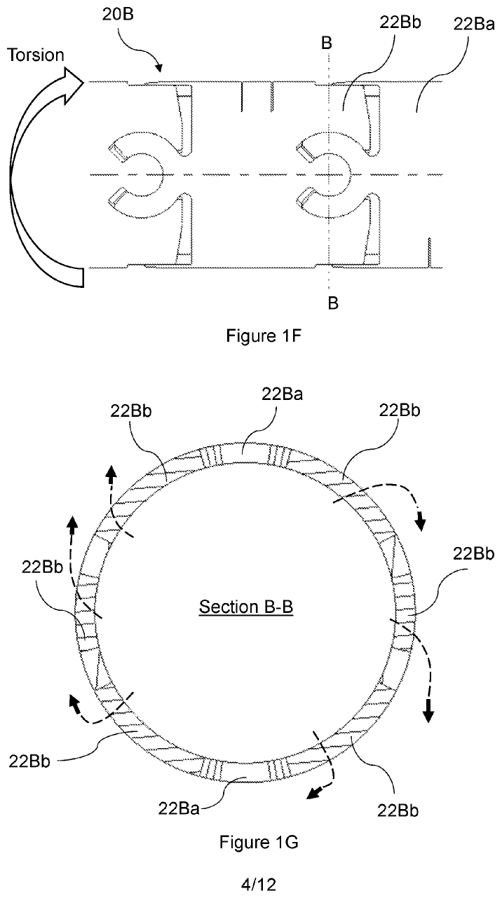

### DETAILED DESCRIPTION

For purposes of brevity and clarity, descriptions of embodiments of the present disclosure are directed to endoscope bending members in accordance with the drawings. While parts of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of features of particular embodiments. In a number of instances, well-known systems, methods, procedures, and components have not been described in detail so as to not unnecessarily obscure features of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment. As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The terms “first”, “second”, etc. are used merely as labels or identifiers and are not intended to impose numerical requirements on their associated terms.

Representative or exemplary embodiments of the present disclosure describe bending members for endoscopes 10. In many embodiments, the endoscope 10 includes an operation handle 12 and an insertion tube 14, and the insertion tube 14 includes a bending member at a distal end 16 of the insertion tube 14. The insertion tube 14 may include a lighting device for illumination of the affected area and image sensor for providing an image of the affected area, such as the duodenum or ureters of the patient, thereby allowing doctors to observe details of the affected area. The insertion tube 14 may accommodate accessories such as for cell collection and laser treatment, etc. The insertion tube 14 may be rigid or flexible, but the bending member is controllable and bendable using control or angulation wires in the insertion tube 14. For example, the bending member is configured to bend in top-down and/or left-right directions.

### First Bending Member 100

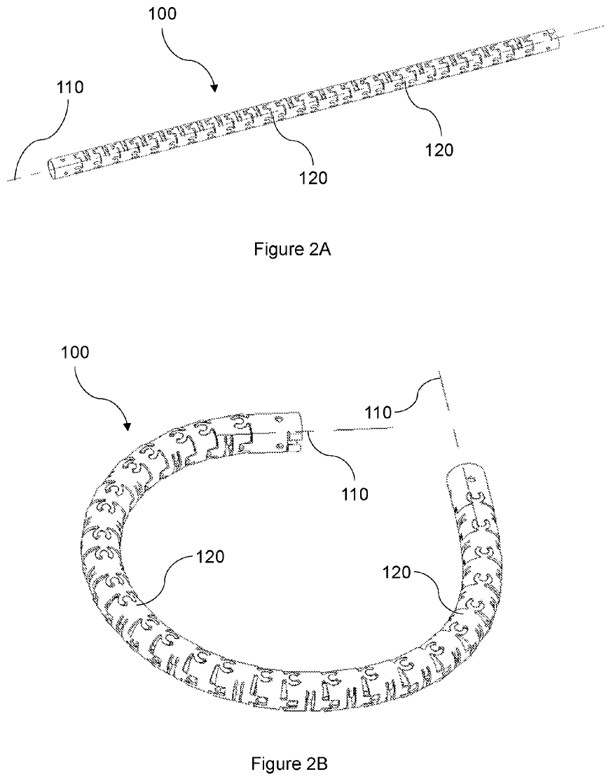

As shown in FIGS. 2A and 2B, some embodiments of the present disclosure describe a first bending member 100 for an endoscope 10, as well as an endoscope 10 comprising the first bending member 100. Specifically, the first bending member 100 is disposed at the distal end 16 of the insertion tube 14. The first bending member 100 includes a lumen extending therethrough. The lumen is configured to accommodate angulation wires for controlling bending of the first bending member 100, as well as other endoscopy components such as lighting devices and image sensors. The first bending member 100 further includes a plurality of bending segments 120 successively and rotatably connected to each other along a longitudinal axis 110 through the lumen such that the first bending member 100 is bendable. Specifically, the individual bending segments 120 are controllable, using the angulation wires, relative to each other to thereby bend the first bending member 100 in the desired direction.

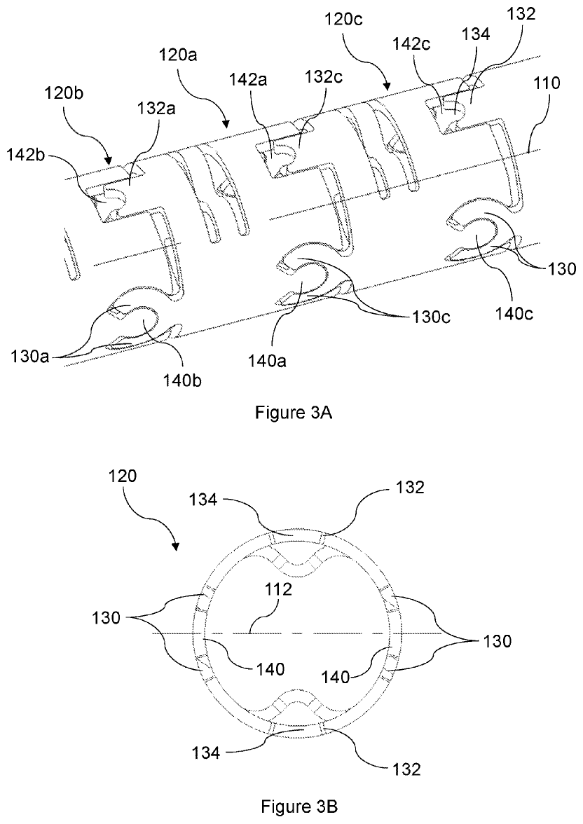

Further as shown in FIGS. 3A and 3B, each bending segment 120 includes a pair of proximal arcuate extensions 130 extending proximally from the bending segment 120, and a proximal stopper element 132 that is circumferentially offset about the longitudinal axis 110 from the pair of proximal arcuate extensions 130. For example, the proximal stopper element 132 is circumferentially offset by 90° about the longitudinal axis 110 from the pair of proximal arcuate extensions 130. It will be appreciated that the proximal stopper element 132 may be circumferentially offset by other angles, such as 45° or 60°.

Each bending segment 120 further includes a distal pivot extension 140 extending distally from the bending segment 120, and a distal stopper element 142 that is circumferentially offset about the longitudinal axis 110 from the distal pivot extension 140. For example, the distal stopper element 142 is circumferentially offset by 90° about the longitudinal axis 110 from the distal pivot extension 140. It will be appreciated that the distal stopper element 142 may be circumferentially offset by other angles, such as 45° or 60°.

As shown in FIG. 3A, a first bending segment 120a (e.g. an nth bending segment) is engaged with a proximally adjacent bending segment 120b (e.g. an n−1th bending segment) and a distally adjacent bending segment 120c (e.g. an n+1th bending segment).

The pair of proximal arcuate extensions 130a of the first bending segment 120a are engaged around the distal pivot extension 140b of the proximally adjacent bending segment 120b, such that the first bending segment 120a and proximally adjacent bending segment 120b are rotatable about a proximal bending axis 112 through the distal pivot extension 140b of the proximally adjacent bending segment 120b. Specifically, the distal pivot extension 140b of the proximally adjacent bending segment 120b is disposed within a concave space between the pair of proximal arcuate extensions 130a of the first bending segment 120a.

The pair of proximal arcuate extensions 130c of the distally adjacent bending segment 120c are engaged around the distal pivot extension 140a of the first bending segment 120a, such that the first bending segment 120a and distally adjacent bending segment 120c are rotatable about a distal bending axis 112 through the distal pivot extension 140a of the first bending segment 120a. Specifically, the distal pivot extension 140a of the first bending segment 120a is disposed within a concave space between the pair of proximal arcuate extensions 130c of the distally adjacent bending segment 120c.

In some embodiments as shown in FIG. 3A, the proximal stopper element 132 includes an extension extending proximally from the bending segment 120, and the distal stopper element 142 includes a recess.

The proximal stopper element 132a (extension) of the first bending segment 120a is engaged with the distal stopper element 142b (recess) of the proximally adjacent bending segment 140b to resist rotation of the first bending segment 120a and proximally adjacent bending segment 120b about the longitudinal axis 110. Specifically, the engaged proximal stopper element 132a (extension) and distal stopper element 142b (recess) counter torsion forces acting on the first bending segment 120a and proximally adjacent bending segment 140b.

The proximal stopper element 132c (extension) of the distally adjacent bending segment 120c is engaged with the distal stopper element 142a (recess) of the first bending segment 120a to resist rotation of the first bending segment 120a and distally adjacent bending segment 120c about the longitudinal axis 110. Specifically, the engaged proximal stopper element 132c (extension) and distal stopper element 142a (recess) counter torsion forces acting on the first bending segment 120a and distally adjacent bending segment 120c.

In some embodiments, the proximal stopper element 132 includes a recess, and the distal stopper element 142 includes an extension extending distally from the bending segment 120. The proximal stopper element 132a (recess) of the first bending segment 120a would be engaged with the distal stopper element 142b (extension) of the proximally adjacent bending segment 140b, and the proximal stopper element 132c (recess) of the distally adjacent bending segment 120c would be engaged with the distal stopper element 142a (extension) of the first bending segment 120a.

Accordingly, the bending segments 120 are rotatable with respect to each other about the respective bending axes 112 to thereby bend the first bending member 100, the bending axes 112 perpendicular to the longitudinal axis 110. Further, the proximal stopper elements 132 and distal stopper elements 142 cooperatively resist rotation of the bending segments 120 about the longitudinal axis 110 by countering torsion forces acting on the first bending member 100.

As shown in FIG. 3B, each bending segment 120 may include another set of the pair of proximal arcuate extensions 130 and the distal pivot extension 140 on the other lateral side of the bending segment 120. The respective bending axes 112 also pass through the respective other distal pivot extensions 140 on the respective other lateral sides.

As shown in FIG. 3B, each bending segment 120 may include another set of the proximal stopper element 132 and the distal stopper element 142 on the other lateral side of the bending segment 120. Both sets of the proximal stopper elements 132 and distal stopper elements 142 are respectively engaged with each other to cooperatively resist rotation of the respective bending segment 120 about the longitudinal axis 110.

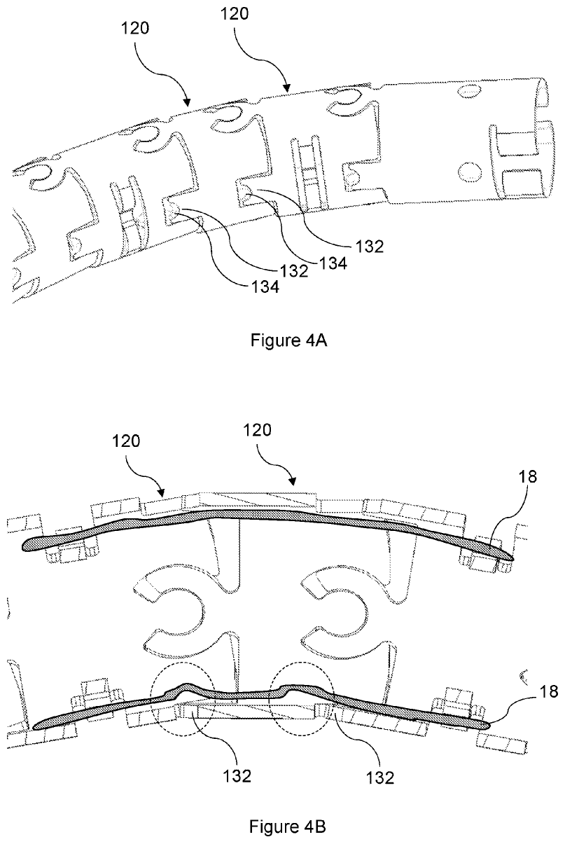

In some embodiments as shown in FIGS. 4A and 4B, the proximal stopper element 132 includes an extension extending proximally from the bending segment 120. Further, the extension of the proximal stopper element 132 includes a cut-out section 134 for accommodating an angulation wire 18 in the lumen during bending of the first bending member 100. It will be appreciated that in some embodiments, the distal stopper element 142 includes an extension extending distally from the bending segment 120, and the extension includes a similar cut-out section 134.

The cut-out section 134 is preferably a concave chamfer, fillet, or recess. For example, when the first bending member 100 is bent, the cut-out section 134 allows for smooth movement of the angulation wire 18 and minimizes contact between the proximal stopper element 132 with the angulation wire 18. Without the cut-out section 134, when the first bending member 100 is bent, the proximal stopper element 132 having a straight end edge would protrude into the lumen and may contact the angulation wire 18. This contact may damage and/or break the angulation wire 18 and/or the first bending member 100, such as shown in the circled areas in FIG. 4B. The cut-out section 134 thus prevents the angulation wire 18 from interfering with the body of the first bending member 100, resulting in smoother bending and better durability.

In some embodiments, the first bending member 100 is bendable in two directions. Specifically, for each bending segment 120, the distal pivot extension 140 is circumferentially aligned to the pair of proximal arcuate extensions 130. For example, the first bending segment 120a is rotatable with respect to the proximally adjacent bending segment 120b about the proximal bending axis 112, and the first bending segment 120a is also rotatable with respect to the distally adjacent bending segment 120c about the distal bending axis 112. The proximal and distal bending axes 112 are parallel to each other and perpendicular to the longitudinal axis 110.

Hence, the bending segments 120 are rotatably connected to each other such that all the bending axes 112 are parallel to each other and the first bending member 100 is bendable in two directions. Specifically, the first bending member 100 is bendable in along a single plane, such as in the top and down directions, or in the left and right directions.

In some embodiments, the first bending member 100 is bendable in four directions. Specifically, for each bending segment 120, the distal pivot extension 140 is circumferentially offset by 90° about the longitudinal axis 110 from the pair of proximal arcuate extensions 130. For example, the first bending segment 120a is rotatable with respect to the proximally adjacent bending segment 120b about the proximal bending axis 112, and the first bending segment 120a is also rotatable with respect to the distally adjacent bending segment 120c about the distal bending axis 112. The proximal and distal bending axes 112 are perpendicular to each other and perpendicular to the longitudinal axis 110.

Hence, the bending segments 120 are rotatably connected to each other such that all the bending axes 112 are mutually perpendicular to each other and the first bending member 100 is bendable in four directions. Specifically, the first bending member 100 is bendable in along two mutually perpendicular planes, such as in the top, down, left, and right directions.

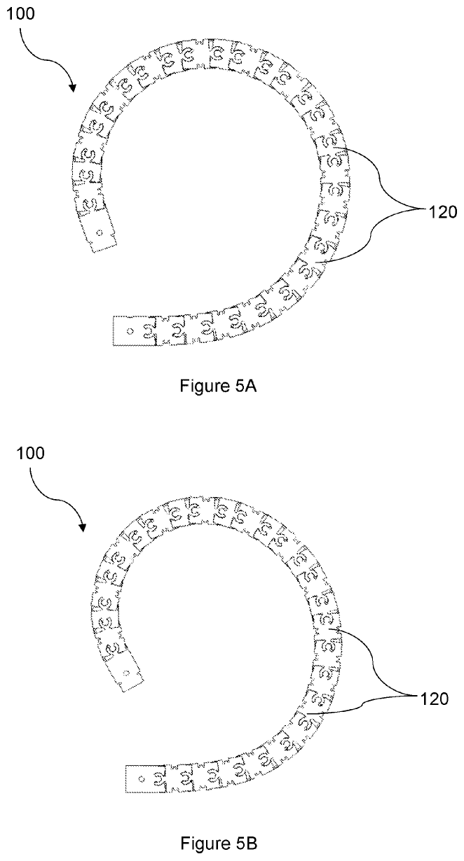

Various features of the bending segments 120 may be changed to adjust the bending shape, bending radius, and/or bending angle of the first bending member 100. For example, dimensions of bending segments 120 and/or the interfacing parts of the bending segments 120 may be changed. For example, some bending segments 120 may be longer while some bending segments 120 may be shorter. Shorter bending segments 120 would result in a smaller bending radius for the first bending member 100. For example as shown in FIG. 3A, the first bending segment 120a is longitudinally shorter than the distally adjacent bending segment 120c. FIGS. 5A and 5B show the first bending member 100 with different bending shapes, bending radii, and bending angles.

### Second Bending Member 200

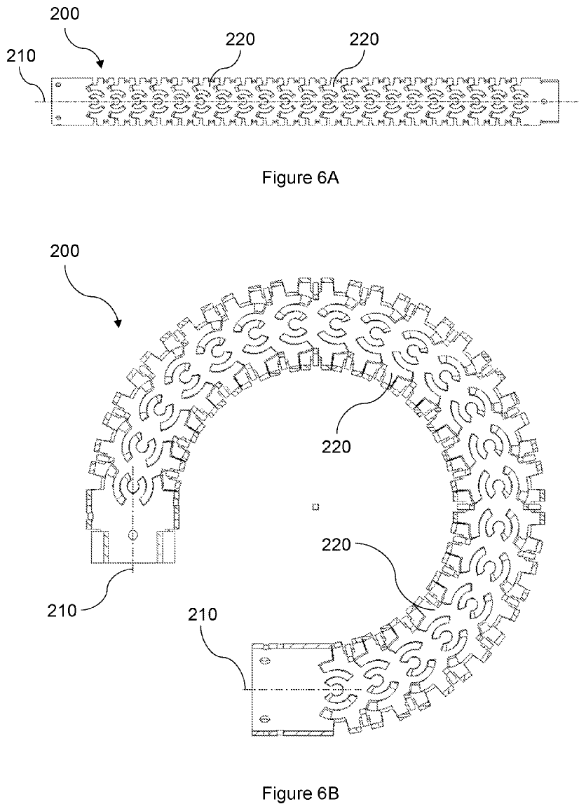

As shown in FIGS. 6A and 6B, some embodiments of the present disclosure describe a second bending member 200 for an endoscope 10, as well as an endoscope 10 comprising the second bending member 200. Specifically, the second bending member 200 is disposed at the distal end 16 of the insertion tube 14. The second bending member 200 includes a lumen extending therethrough. The lumen is configured to accommodate angulation wires for controlling bending of the second bending member 200, as well as other endoscopy components such as lighting devices and image sensors. The second bending member 200 further includes a plurality of bending segments 220 successively and rotatably connected to each other along a longitudinal axis 210 through the lumen such that the second bending member 200 is bendable. Specifically, the individual bending segments 220 are controllable, using the angulation wires, relative to each other to thereby bend the second bending member 200 in the desired direction.

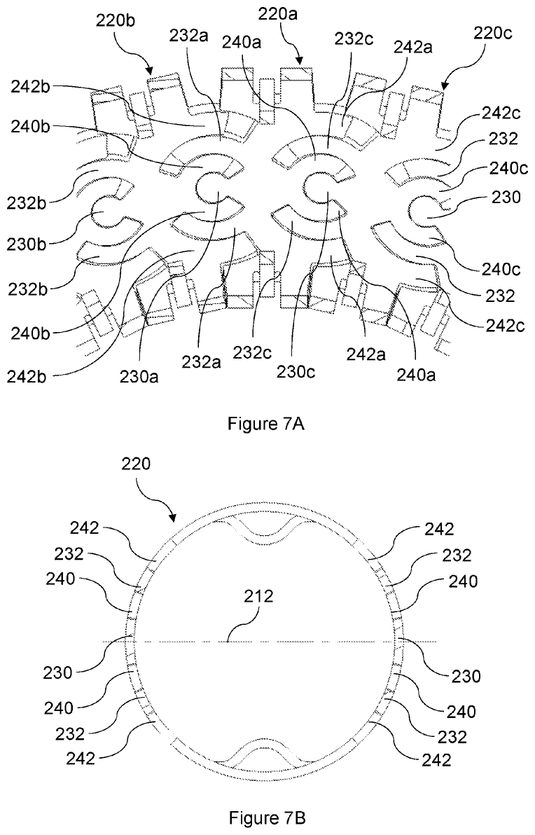

Further as shown in FIGS. 7A and 7B, each bending segment 220 includes a proximal pivot extension 230 extending proximally from the bending segment, and a pair of proximal arcuate extensions 232 extending proximally from the bending segment 220. The proximal pivot extension 230 is disposed between the pair of proximal arcuate extensions 232. Each bending segment 120 further includes an inner pair of distal arcuate extensions 240 extending distally from the bending segment 220, and an outer pair of distal arcuate extensions 242 extending distally from the bending segment 220. The inner pair of distal arcuate extensions 240 is disposed between the outer pair of distal arcuate extensions 242.

As shown in FIG. 7A, a first bending segment 220a (e.g. an nth bending segment) is engaged with a proximally adjacent bending segment 220b (e.g. an n−1th bending segment) and a distally adjacent bending segment 220c (e.g. an n+1th bending segment).

The inner pair of distal arcuate extensions 240a of the first bending segment 220a are engaged around the proximal pivot extension 230c of the distally adjacent bending segment 220c. Specifically, the proximal pivot extension 230c of the distally adjacent bending segment 220c is disposed within a concave space between the inner pair of distal arcuate extensions 240a of the first bending segment 220a.

The pair of proximal arcuate extensions 232c of the distally adjacent bending segment 220c are engaged around the inner pair of distal arcuate extensions 240a of the first bending segment 220a. Specifically, the inner pair of distal arcuate extensions 240a of the first bending segment 220a and the proximal pivot extension 230c of the distally adjacent bending segment 220c are disposed within a concave space between the pair of proximal arcuate extensions 232c of the distally adjacent bending segment 220c.

The outer pair of distal arcuate extensions 242a of the first bending segment 220a are engaged around the pair of proximal arcuate extensions 232c of the distally adjacent bending segment 220c. Specifically, the pair of proximal arcuate extensions 232c of the distally adjacent bending segment 220c, the inner pair of distal arcuate extensions 240a of the first bending segment 220a, and the proximal pivot extension 230c of the distally adjacent bending segment 220c are disposed within a concave space between the outer pair of distal arcuate extensions 242a of the first bending segment 220a.

The first bending segment 220a and distally adjacent bending segment 220c are rotatably connected to each other, such that the first bending segment 220a and distally adjacent bending segment 220c are rotatable about a distal bending axis 212 through the proximal pivot extension 230c of the distally adjacent bending segment 220c.

The inner pair of distal arcuate extensions 240b of the proximally adjacent bending segment 220b are engaged around the proximal pivot extension 230a of the first bending segment 220a. Specifically, the proximal pivot extension 230a of the first bending segment 220a is disposed within a concave space between the inner pair of distal arcuate extensions 240b of the proximally adjacent bending segment 220b.

The pair of proximal arcuate extensions 232a of the first bending segment 220a are engaged around the inner pair of distal arcuate extensions 240b of the proximally adjacent bending segment 220b. Specifically, the inner pair of distal arcuate extensions 240b of the proximally adjacent bending segment 220b and the proximal pivot extension 230a of the first bending segment 220a are disposed within a concave space between the proximal arcuate extensions 232a of the first bending segment 220a.

The outer pair of distal arcuate extensions 242b of the proximally adjacent bending segment 220b are engaged around the pair of proximal arcuate extensions 232a of the first bending segment 220a. Specifically, the pair of proximal arcuate extensions 232a of the first bending segment 220a, the inner pair of distal arcuate extensions 240b of the proximally adjacent bending segment 220b, and the proximal pivot extension 230a of the first bending segment 220a are disposed within a concave space between the outer pair of distal arcuate extensions 242b of the proximally adjacent bending segment 220b.

The first bending segment 220a and proximally adjacent bending segment 220b are rotatably connected to each other, such that the first bending segment 220a and proximally adjacent bending segment 220b are rotatable about a proximal bending axis 212 through the proximal pivot extension 230a of the first bending segment 220a.

Accordingly, the bending segments 220 are rotatable with respect to each other about the respective bending axes 212 to thereby bend the second bending member 200, the bending axes 212 perpendicular to the longitudinal axis 210.

As shown in FIG. 7B, each bending segment 220 may include another set of the proximal pivot extension 230, the pair of proximal arcuate extensions 232, the inner pair of distal arcuate extensions 240, and the outer pair of distal arcuate extensions 242 on the other lateral side of the bending segment 220. The respective bending axes 212 also pass through the respective other proximal pivot extensions 230 on the respective other lateral sides.

In some embodiments, the second bending member 200 is bendable in two directions. Specifically, for each bending segment 220, the proximal pivot extension 230 is circumferentially aligned to the inner pair of distal arcuate extensions 240. For example, the first bending segment 220a is rotatable with respect to the proximally adjacent bending segment 220b about the proximal bending axis 212, and the first bending segment 220a is also rotatable with respect to the distally adjacent bending segment 220c about the distal bending axis 212. The proximal and distal bending axes 212 are parallel to each other and perpendicular to the longitudinal axis 210.

Hence, the bending segments 220 are rotatably connected to each other such that all the bending axes 212 are parallel to each other and the second bending member 200 is bendable in two directions. Specifically, the second bending member 200 is bendable in along a single plane, such as in the top and down directions, or in the left and right directions.

In some embodiments, the second bending member 200 is bendable in four directions.

Specifically, for each bending segment 220, the proximal pivot extension 230 is circumferentially offset by 90° about the longitudinal axis 210 from the inner pair of distal arcuate extensions 240. For example, the first bending segment 220a is rotatable with respect to the proximally adjacent bending segment 220b about the proximal bending axis 212, and the first bending segment 220a is also rotatable with respect to the distally adjacent bending segment 220c about the distal bending axis 212. The proximal and distal bending axes 212 are perpendicular to each other and perpendicular to the longitudinal axis 210.

Hence, the bending segments 220 are rotatably connected to each other such that all the bending axes 212 are mutually perpendicular to each other and the second bending member 200 is bendable in four directions. Specifically, the second bending member 200 is bendable in along two mutually perpendicular planes, such as in the top, down, left, and right directions.

Various features of the bending segments 220 may be changed to adjust the bending shape, bending radius, and/or bending angle of the second bending member 200. For example, dimensions of bending segments 220 and/or the interfacing parts of the bending segments 220 may be changed. For example, some bending segments 220 may be longer while some bending segments 220 may be shorter. Shorter bending segments 220 would result in a smaller bending radius for the second bending member 200.

In some embodiments, the bending segments 220 of the second bending member 200 may include stopper elements similar to the proximal stopper elements 132 and distal stopper elements 142 of the first bending member 100. Specifically, each bending segment 220 includes a proximal stopper element circumferentially offset about the longitudinal axis 220 from the proximal pivot extension 230, and a distal stopper element circumferentially offset about the longitudinal axis 220 from the inner pair of distal arcuate extensions 240. The circumferential offset preferably 90°, but may be other angles such as 45° or 60°.

The proximal stopper element of the first bending segment 220a is engaged with the distal stopper element of the proximally adjacent bending segment 220b to resist rotation of the first bending segment 220a and proximally adjacent bending segment 220b about the longitudinal axis 210. The proximal stopper element of the distally adjacent bending segment 220c is engaged with the distal stopper element of the first bending segment 220a to resist rotation of the first bending segment 220a and distally adjacent bending segment 220c about the longitudinal axis 210.

It will be appreciated that aspects of the proximal stopper elements 132 and distal stopper elements 142 described above for the first bending member 100, such as the cut-out section 134, apply equally to the proximal stopper elements and distal stopper elements of the second bending member 200.

### Comparison of Bending Members

Experiments were performed on the conventional bending members 20A,20B, the first bending member 100, and the second bending member 200 to evaluate their resistance to torsion forces. The experiments were performed on these bending members with outer diameters of 3 mm and 6 mm. For outer diameters of 3 mm, the conventional bending members 20A,20B can tolerate torsion forces of up to about 1.4 kg and 2.2 kg, respectively, whereas the first bending member 100 and second bending member 200 can tolerate torsion forces of up to about 3.2 kg and 3.6 kg, respectively. For outer diameters of 6 mm, the conventional bending members 20A,20B can tolerate torsion forces of up to about 1.7 kg and 2.8 kg, respectively, whereas the first bending member 100 and second bending member 200 can tolerate torsion forces of up to about 3.8 kg and 4.2 kg, respectively.

The first bending member 100 is able to withstand stronger torsion forces than the conventional bending members 20A,20B. This is because of the proximal stopper elements 132 and distal stopper elements 142 that cooperatively resist rotation of the bending segments 120 about the longitudinal axis 110 and counter torsion forces acting on the first bending member 100.

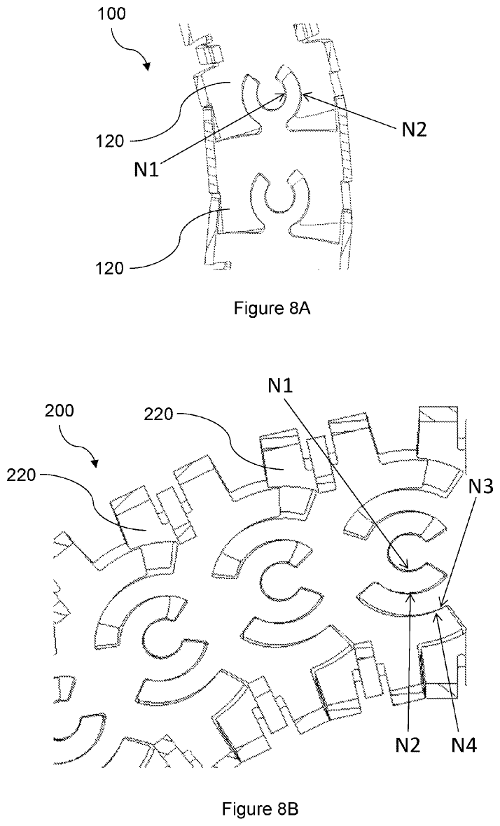

Further, the second bending member 200 is able to withstand stronger torsion forces than the conventional bending members 20A,20B and the first bending member 100. This is because of the twin pairs of distal arcuate extensions 240,242 that enable each bending segment 220 to be more strongly engaged with the adjacent bending segment 220. As shown in FIGS. 8A and 8B, there are two sets of interfacing surfaces (N1,N2) in the engagement between the bending segments 110 of the first bending member 100, whereas there are four sets of interfacing surfaces (N1,N2, N3,N4) in the engagement between the bending segments 210 of the second bending member 200.

The first bending member 100 and second bending member 200 described in various embodiments herein advantageously exhibit high resistance to forces acting on them, specifically bending, torsion, and tension forces, thereby minimizing minimize the risk of damage or breakage of the bending members 100,200, especially during medical procedures which would be dangerous or even fatal to the patient.

The first bending member 100 and second bending member 200 may be manufactured using various manufacturing methods. In some embodiments, the bending segments 120,220 of the bending members 100,200 are individually manufactured, such as by moulding, machining, milling, etc., and the bending segments 120,220 are assembled together to form the bending members 100,200. In some embodiments, the first bending member 100 and second bending member 200 are manufactured by a subtractive manufacturing process such as laser cutting.

### Manufacturing Method 300

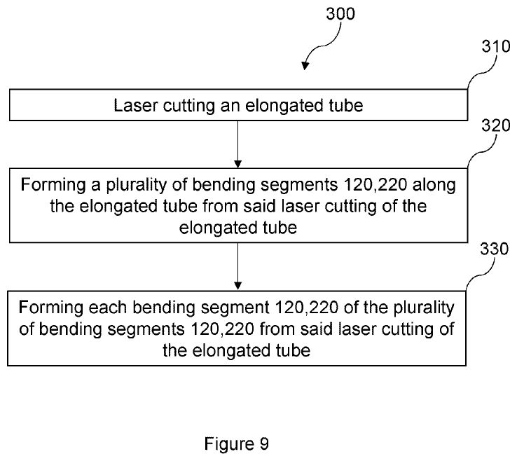

With reference to FIG. 9, various embodiments of the present disclosure describe a method 300 for manufacturing a bending member 100,200 for an endoscope 10 using laser cutting. The method 300 includes a step 310 of laser cutting an elongated tube having a lumen extending therethrough. The elongated tube is a rigid tube that may be made of high strength material, such as stainless steel or surgical steel.

The method 300 includes a step 320 of forming a plurality of bending segments 120,220 along the elongated tube from said laser cutting of the elongated tube, the bending segments 120,220 successively and rotatably connected to each other along a longitudinal axis 110,210 through the lumen such that the bending member 100,200 is bendable. The method 300 includes a step 330 of forming each bending segment 120,220 of the plurality of bending segments 120,220 from said laser cutting of the elongated tube. Various aspects of each bending segment 120,220 are described above and are not further elaborated here for purpose of brevity.

During laser cutting of the elongated tube, a laser beam irradiates radially towards the outer surface of the elongated tube to form the successively connected plurality of bending segments 120,220. Specifically, the laser beam cuts the proximal and distal ends of each bending segment 120,220 to form the respective extensions and concave spaces that engage adjacent bending segments 120,220. Gaps between the bending segments 120,220 may range from 0.1 mm to 0.3 mm, such as 0.2 mm to 0.3 mm, 0.2 mm to 0.25 mm, or 0.15 mm to 0.25 mm. Laser cutting of the elongated tube using the radially-directed laser beam to form the bending segments 120,220 advantageously forms a natural locking between the bending segments 120,220 and prevents the individual bending segments 120,220 from becoming disconnected.

### Additive Manufacturing

In some embodiments, the bending members 100,200 or parts thereof, such as the bending segments 120,220, may be manufactured using an additive manufacturing process. A common example of additive manufacturing is three-dimensional (3D) printing; however, other methods of additive manufacturing are available. Rapid prototyping or rapid manufacturing are also terms which may be used to describe additive manufacturing processes.

As used herein, “additive manufacturing” refers generally to manufacturing processes wherein successive layers of material(s) are provided on each other to “build-up” layer-by-layer or “additively fabricate”, a 3D component. This is compared to some subtractive manufacturing methods (such as cutting, milling, or drilling), wherein material is successively removed to fabricate the part. The successive layers generally fuse together to form a monolithic component which may have a variety of integral sub-components. In particular, the manufacturing process may allow an example of the disclosure to be integrally formed and include a variety of features not possible when using prior manufacturing methods.

Additive manufacturing methods described herein enable manufacture to any suitable size and shape with various features which may not have been possible using prior manufacturing methods. Additive manufacturing can create complex geometries without the use of any sort of tools, moulds, or fixtures, and with little or no waste material. Instead of machining components from solid billets of plastic or metal, much of which is cut away and discarded, the only material used in additive manufacturing is what is required to shape the part.

Suitable additive manufacturing techniques in accordance with the present disclosure include, for example, Fused Deposition Modelling (FDM), Selective Laser Sintering (SLS), 3D printing such as by inkjets and laserjets, Stereolithography (SLA), Direct Selective Laser Sintering (DSLS), Electron Beam Sintering (EBS), Electron Beam Melting (EBM), Laser Engineered Net Shaping (LENS), Electron Beam Additive Manufacturing (EBAM), Laser Net Shape Manufacturing (LNSM), Direct Metal Deposition (DMD), Digital Light Processing (DLP), Continuous Digital Light Processing (CDLP), Direct Selective Laser Melting (DSLM), Selective Laser Melting (SLM), Direct Metal Laser Melting (DMLM), Direct Metal Laser Sintering (DMLS), Material Jetting (MJ), NanoParticle Jetting (NPJ), Drop On Demand (DOD), Binder Jetting (BJ), Multi Jet Fusion (MJF), Laminated Object Manufacturing (LOM), and other known processes.

The additive manufacturing processes described herein may be used for forming components using any suitable material. For example, the material may be metal, plastic, polymer, composite, or any other suitable material that may be in solid, liquid, powder, sheet material, wire, or any other suitable form or combinations thereof. More specifically, according to exemplary embodiments of the present disclosure, the additively manufactured components described herein may be formed in part, in whole, or in some combination of materials suitable for use in additive manufacturing processes and which may be suitable for the fabrication of examples described herein.

As noted above, the additive manufacturing process disclosed herein allows a single component to be formed from multiple materials. Thus, the examples described herein may be formed from any suitable mixtures of the above materials. For example, a component may include multiple layers, segments, or parts that are formed using different materials, processes, and/or on different additive manufacturing machines. In this manner, components may be constructed which have different materials and material properties for meeting the demands of any particular application. In addition, although the components described herein are constructed entirely by additive manufacturing processes, it should be appreciated that in alternate embodiments, all or a portion of these components may be formed via casting, machining, and/or any other suitable manufacturing process. Indeed, any suitable combination of materials and manufacturing methods may be used to form these components.

Additive manufacturing processes typically fabricate components based on 3D information, for example a 3D computer model (or design file), of the component. Accordingly, examples described herein not only include products or components as described herein, but also methods of manufacturing such products or components via additive manufacturing and computer software, firmware or hardware for controlling the manufacture of such products via additive manufacturing.

The structure of the product may be represented digitally in the form of a design file. A design file, or computer aided design (CAD) file, is a configuration file that encodes one or more of the surface or volumetric configuration of the shape of the product. That is, a design file represents the geometrical arrangement or shape of the product.

Design files can take any now known or later developed file format. For example, design files may be in the Stereolithography or “Standard Tessellation Language” (.stl) format which was created for Stereolithography CAD programs of 3D Systems, or the Additive Manufacturing File (.amf) format, which is an American Society of Mechanical Engineers (ASME) standard that is an extensible markup-language (XML) based format designed to allow any CAD software to describe the shape and composition of any 3D object to be fabricated on any additive manufacturing printer. Further examples of design file formats include AutoCAD (.dwg) files, Blender (.blend) files, Parasolid (.x_t) files, 3D Manufacturing Format (0.3mf) files, Autodesk (3ds) files, Collada (.dae) files and Wavefront (obj) files, although many other file formats exist.

Design files can be produced using modelling (e.g. CAD modelling) software and/or through scanning the surface of a product to measure the surface configuration of the product. Once obtained, a design file may be converted into a set of computer executable instructions that, once executed by a processer, cause the processor to control an additive manufacturing apparatus to produce a product according to the geometrical arrangement specified in the design file. The conversion may convert the design file into slices or layers that are to be formed sequentially by the additive manufacturing apparatus. The instructions (otherwise known as geometric code or “G-code”) may be calibrated to the specific additive manufacturing apparatus and may specify the precise location and amount of material that is to be formed at each stage in the manufacturing process. As discussed above, the formation may be through deposition, through sintering, or through any other form of additive manufacturing method.

The code or instructions may be translated between different formats, converted into a set of data signals and transmitted, received as a set of data signals and converted to code, stored, etc., as necessary. The instructions may be an input to the additive manufacturing system and may come from a part designer, an intellectual property (IP) provider, a design company, the operator or owner of the additive manufacturing system, or from other sources. An additive manufacturing system may execute the instructions to fabricate the product using any of the technologies or methods disclosed herein.

Design files or computer executable instructions may be stored in a (transitory or non-transitory) computer readable storage medium (e.g., memory, storage system, etc.)

storing code, or computer readable instructions, representative of the product to be produced. As noted, the code or computer readable instructions defining the product that can be used to physically generate the object, upon execution of the code or instructions by an additive manufacturing system. For example, the instructions may include a precisely defined 3D model of the product and can be generated from any of a large variety of well-known CAD software systems such as AutoCAD®, TurboCADR, DesignCAD 3D Max, etc. Alternatively, a model or prototype of the product may be scanned to determine the 3D information of the product. Accordingly, by controlling an additive manufacturing apparatus according to the computer executable instructions, the additive manufacturing apparatus can be instructed to print out the product.

In light of the above, embodiments include methods of manufacture via additive manufacturing. This includes the steps of obtaining a design file representing the product and instructing an additive manufacturing apparatus to manufacture the product according to the design file. The additive manufacturing apparatus may include a processor that is configured to automatically convert the design file into computer executable instructions for controlling the manufacture of the product. In these embodiments, the design file itself can automatically cause the production of the product once input into the additive manufacturing apparatus. Accordingly, in this embodiment, the design file itself may be considered computer executable instructions that cause the additive manufacturing apparatus to manufacture the product. Alternatively, the design file may be converted into instructions by an external computing system, with the resulting computer executable instructions being provided to the additive manufacturing apparatus.

Given the above, the design and manufacture of implementations of the subject matter and the operations described in this specification can be realized using digital electronic circuitry, or in computer software, firmware, or hardware, including the structures disclosed in this specification and their structural equivalents, or in combinations of one or more of them. For instance, hardware may include processors, microprocessors, electronic circuitry, electronic components, integrated circuits, etc.

Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them.

Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

Although additive manufacturing technology is described herein as enabling fabrication of complex objects by building objects point-by-point, layer-by-layer, typically in a vertical direction, other methods of fabrication are possible and within the scope of the present subject matter. For example, although the discussion herein refers to the addition of material to form successive layers, one skilled in the art will appreciate that the methods and structures disclosed herein may be practiced with any additive manufacturing technique or other manufacturing technology.

In the foregoing detailed description, embodiments of the present disclosure in relation to endoscope bending members are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A bending member for an endoscope, the bending member comprising:
a lumen extending therethrough;
a plurality of bending segments successively and rotatably connected to each other along a longitudinal axis through the lumen such that the bending member is bendable; and
each bending segment comprising:
a pair of proximal arcuate extensions extending proximally from the bending segment;
a proximal stopper element circumferentially offset about the longitudinal axis from the pair of proximal arcuate extensions; and
a distal pivot extension extending distally from the bending segment; and
a distal stopper element circumferentially offset about the longitudinal axis from the distal pivot extension,
wherein a first bending segment is engaged with a proximally adjacent bending segment and a distally adjacent bending segment, such that:
the pair of proximal arcuate extensions of the first bending segment are engaged around the distal pivot extension of the proximally adjacent bending segment, such that the first and proximally adjacent bending segments are rotatable about a proximal bending axis through the distal pivot extension of the proximally adjacent bending segment;
the pair of proximal arcuate extensions of the distally adjacent bending segment are engaged around the distal pivot extension of the first bending segment, such that the first and distally adjacent bending segments are rotatable about a distal bending axis through the distal pivot extension of the first bending segment;
the proximal element of the first bending segment is engaged with the distal stopper element of the proximally adjacent bending segment to resist rotation of the first and proximally adjacent bending segments about the longitudinal axis; and
the proximal stopper element of the distally adjacent bending segment is engaged with the distal stopper element of the first bending segment to resist rotation of the first and distally adjacent bending segments about the longitudinal axis; and
wherein bending segments are rotatable with respect to each other about the respective bending axes to thereby bend the bending member, the bending axes perpendicular to the longitudinal axis.

2. The bending member according to claim 1, wherein:
the proximal stopper element comprises an extension extending proximally from the bending segment;
the distal stopper element comprises a recess.

3. The bending member according to claim 2, wherein the extension of the proximal stopper element comprises a cut-out section for accommodating an angulation wire in the lumen.

4. The bending member according to claim 1, wherein:
the proximal stopper element comprises a recess; and
the distal stopper element comprises an extension extending distally from the bending segment.

5. The bending member according to claim 4, wherein the extension of the distal stopper element comprises a cut-out section for accommodating an angulation wire in the lumen.

6. The bending member according to claim 1, wherein:
the proximal stopper element is circumferentially offset by 90° about the longitudinal axis from the pair of proximal arcuate extensions; and
the distal stopper element is circumferentially offset by 90° about the longitudinal axis from the distal pivot extension.

7. The bending member according to to claim 1, wherein the distal pivot extension is circumferentially aligned to the pair of proximal arcuate extensions, such that the bending member is bendable in two directions.

8. The bending member according to claim 1, wherein the distal pivot extension is circumferentially offset by 90° about the longitudinal axis from the pair of proximal arcuate extensions, such that the bending member is bendable in four directions.

9. (canceled)

10. An endoscope comprising a bending member according to claim 1.

11. (canceled)

12. (canceled)

13. A method for manufacturing a bending member for an endoscope, the method comprising:
laser cutting an elongated tube comprising a lumen extending therethrough;
forming a plurality of bending segments along the elongated tube from said laser cutting of the elongated tube, the bending segments successively and rotatably connected to each other along a longitudinal axis through the lumen such that the bending member is bendable; and
forming each bending segment of the plurality of bending segments from said laser cutting of the elongated tube, each bending segment comprising:
a pair of proximal arcuate extensions extending proximally from the bending segment;
a proximal stopper element circumferentially offset about the longitudinal axis from the pair of proximal arcuate extensions; and
a distal pivot extension extending distally from the bending segment; and
a distal stopper element circumferentially offset about the longitudinal axis from the distal pivot extension,
wherein a first bending segment is engaged with a proximally adjacent bending segment and a distally adjacent bending segment, such that:
the pair of proximal arcuate extensions of the first bending segment are engaged around the distal pivot extension of the proximally adjacent bending segment, such that the first and proximally adjacent bending segments are rotatable about a proximal bending axis through the distal pivot extension of the proximally adjacent bending segment;
the pair of proximal arcuate extensions of the distally adjacent bending segment are engaged around the distal pivot extension of the first bending segment, such that the first and distally adjacent bending segments are rotatable about a distal bending axis through the distal pivot extension of the first bending segment;
the proximal element of the first bending segment is engaged with the distal stopper element of the proximally adjacent bending segment to resist rotation of the first and proximally adjacent bending segments about the longitudinal axis; and
the proximal stopper element of the distally adjacent bending segment is engaged with the distal stopper element of the first bending segment to resist rotation of the first and distally adjacent bending segments about the longitudinal axis; and
wherein bending segments are rotatable with respect to each other about the respective bending axes to thereby bend the bending member, the bending axes perpendicular to the longitudinal axis.

14. (canceled)

15. (canceled)

16. (canceled)

17. (canceled)

18. (canceled)

19. (canceled)

20. (canceled)

21. A bending member for an endoscope, the bending member comprising:
a lumen extending therethrough;
a plurality of bending segments successively and rotatably connected to each other along a longitudinal axis through the lumen such that the bending member is bendable; and
each bending segment comprising:
a proximal pivot extension extending proximally from the bending segment;
a pair of proximal arcuate extensions extending proximally from the bending segment, the proximal pivot extension disposed between the pair of proximal arcuate extensions;
an inner pair of distal arcuate extensions extending distally from the bending segment; and
an outer pair of distal arcuate extensions extending distally from the bending segment, the inner pair of distal arcuate extensions disposed between the outer pair of distal arcuate extensions,
wherein a first bending segment is engaged with a proximally adjacent bending segment and a distally adjacent bending segment, such that:
the inner pair of distal arcuate extensions of the first bending segment are engaged around the proximal pivot extension of the distally adjacent bending segment, the pair of proximal arcuate extensions of the distally adjacent bending segment are engaged around the inner pair of distal arcuate extensions of the first bending segment, and the outer pair of distal arcuate extensions of the first bending segment are engaged around the pair of proximal arcuate extensions of the distally adjacent bending segment, such that the first and distally adjacent bending segments are rotatable about a distal bending axis through the proximal pivot extension of the distally adjacent bending segment; and
the inner pair of distal arcuate extensions of the proximally adjacent bending segment are engaged around the proximal pivot extension of the first bending segment, the pair of proximal arcuate extensions of the first bending segment are engaged around the inner pair of distal arcuate extensions of the proximally adjacent bending segment, and the outer pair of distal arcuate extensions of the proximally adjacent bending segment are engaged around the pair of proximal arcuate extensions of the first bending segment, such that the first and proximally adjacent bending segments are rotatable about a proximal bending axis through the proximal pivot extension of the first bending segment; and
wherein bending segments are rotatable with respect to each other about the respective bending axes to thereby bend the bending member, the bending axes perpendicular to the longitudinal axis.

22. The bending member according to claim 21, wherein the inner pair of distal arcuate extensions are circumferentially aligned to the proximal pivot extension, such that the bending member is bendable in two directions.

23. The bending member according to claim 21, wherein the inner pair of distal arcuate extensions are circumferentially offset by 90° about the longitudinal axis from the proximal pivot extension, such that the bending member is bendable in four directions.

24. The bending member according to claim 21, wherein:
each bending segment further comprises:
a proximal stopper element circumferentially offset about the longitudinal axis from the proximal pivot extension; and
a distal stopper element circumferentially offset about the longitudinal axis from the inner pair of distal arcuate extensions;
the proximal element of the first bending segment is engaged with the distal stopper element of the proximally adjacent bending segment to resist rotation of the first and proximally adjacent bending segments about the longitudinal axis; and
the proximal stopper element of the distally adjacent bending segment is engaged with the distal stopper element of the first bending segment to resist rotation of the first and distally adjacent bending segments about the longitudinal axis.

25. The bending member according to claim 24, wherein:
the proximal stopper element comprises an extension extending proximally from the bending segment;
the distal stopper element comprises a recess.

26. The bending member according to claim 25, wherein the extension of the proximal stopper element comprises a cut-out section for accommodating an angulation wire in the lumen.

27. The bending member according to claim 24, wherein:
the proximal stopper element comprises a recess; and
the distal stopper element comprises an extension extending distally from the bending segment.

28. The bending member according to claim 25, wherein the extension of the distal stopper element comprises a cut-out section for accommodating an angulation wire in the lumen.

29. The bending member according to claim 24, wherein:
the proximal stopper element is circumferentially offset by 90° about the longitudinal axis from the pair of proximal arcuate extensions; and
the distal stopper element is circumferentially offset by 90° about the longitudinal axis from the distal pivot extension.

30. (canceled)

31. An endoscope comprising a bending member according to claim 21.

32. (canceled)

33. (canceled)

34. A method for manufacturing a bending member for an endoscope, the method comprising:
laser cutting an elongated tube comprising a lumen extending therethrough;
forming a plurality of bending segments along the elongated tube from said laser cutting of the elongated tube, the bending segments successively and rotatably connected to each other along a longitudinal axis through the lumen such that the bending member is bendable; and
forming each bending segment of the plurality of bending segments from said laser cutting of the elongated tube, each bending segment comprising:
a proximal pivot extension extending proximally from the bending segment;
a pair of proximal arcuate extensions extending proximally from the bending segment, the proximal pivot extension disposed between the pair of proximal arcuate extensions;
an inner pair of distal arcuate extensions extending distally from the bending segment; and
an outer pair of distal arcuate extensions extending distally from the bending segment, the inner pair of distal arcuate extensions disposed between the outer pair of distal arcuate extensions,
wherein a first bending segment is engaged with a proximally adjacent bending segment and a distally adjacent bending segment, such that:
the inner pair of distal arcuate extensions of the first bending segment are engaged around the proximal pivot extension of the distally adjacent bending segment, the pair of proximal arcuate extensions of the distally adjacent bending segment are engaged around the inner pair of distal arcuate extensions of the first bending segment, and the outer pair of distal arcuate extensions of the first bending segment are engaged around the pair of proximal arcuate extensions of the distally adjacent bending segment, such that the first and distally adjacent bending segments are rotatable about a distal bending axis through the proximal pivot extension of the distally adjacent bending segment; and
the inner pair of distal arcuate extensions of the proximally adjacent bending segment are engaged around the proximal pivot extension of the first bending segment, the pair of proximal arcuate extensions of the first bending segment are engaged around the inner pair of distal arcuate extensions of the proximally adjacent bending segment, and the outer pair of distal arcuate extensions of the proximally adjacent bending segment are engaged around the pair of proximal arcuate extensions of the first bending segment, such that the first and proximally adjacent bending segments are rotatable about a proximal bending axis through the proximal pivot extension of the first bending segment; and
wherein bending segments are rotatable with respect to each other about the respective bending axes to thereby bend the bending member, the bending axes perpendicular to the longitudinal axis.

35. (canceled)

36. (canceled)

37. (canceled)

38. (canceled)

39. (canceled)

40. (canceled)

41. (canceled)

42. (canceled)
